(19) United States
(12) Patent Application Publication
(10) Pub. No.: US 2021/0137038 A1
(43) Pub. Date: May 13, 2021

(54) A PLANT POLLINATION CONTROL BAG

(71) Applicant: Duraweld Limited, Yorkshire (GB)

(72) Inventors: Derek PALEY, Yorkshire (GB); Hannah SENIOR, Yorkshire (GB); Stuart CARTER, Yorkshire (GB)

(21) Appl. No.: 16/476,533

(22) PCT Filed: Jan. 10, 2018
(86) PCT No.: PCT/GB2018/050064

§ 371 Date: Jul. 8, 2019

(30) Foreign Application Priority Data
Jan. 10, 2017 (GB) 1700434.2

(51) Int. Cl. A01H 1/02; A01G 13/04

(52) U.S. Cl. CPC A01H 1/027; A01G 2013/046; A01G 13/043

(57) ABSTRACT

A plant pollination control bag (10) defines, in an expanded condition, a space (12) in which a plant part is receivable. The bag (10) defines an opening (14) through which the plant part passes into the space (12). In the expanded condition, the bag (10) has an end face (16) which is substantially square or rectangular in shape.

### BACKGROUND OF THE INVENTION

### 1. Field of the Invention

The present invention relates to a plant pollination control bag.

### 2. The Prior Art

In plant science, it is desirable to be able to control plant pollination. It is known to use bags for this purpose, which are located over a reproductive part of the plant and provide a barrier to the movement of pollen. However, fitting the bags over the reproductive part can cause damage if the bag has not been properly opened, or has collapsed after opening.

### SUMMARY OF THE INVENTION

According to a first aspect of the present invention, there is provided a plant pollination control bag, wherein, in an expanded condition, the bag defines a space in which a plant part is receivable, the bag defining an opening through which the plant part passes into the space, wherein, in the expanded condition, the bag has an end face which is substantially square or rectangular in shape.

Possibly, the end face is opposite the opening, and, in the expanded condition, may lie in a plane parallel to the opening.

Possibly, the bag is movable between the expanded condition and a flat condition, in which the bag is substantially flat.

Possibly, the bag includes reinforcement arrangements, which may hold the bag in the expanded condition. Possibly, the bag includes two reinforcement arrangements, which may be located on opposite sides of the bag. Possibly, each reinforcement arrangement reinforces an opposite reinforcement edge of the end face.

Possibly, each reinforcement arrangement includes a flap, which may fold outwardly from the respective reinforced edge of the end face, possibly away from the end face towards a side face of the bag.

Possibly, each reinforcement arrangement includes a fastening, which may fasten the flap to the respective side face of the bag. The fastening may comprise a weld, adhesive, a rivet, a staple or a stud, and may be formed by welding, gluing, riveting, stapling or studding.

Possibly, in the expanded condition, the bag comprises four side faces. Possibly, in the flat condition, the bag comprises two side faces.

Possibly, in the flat condition, the end face is coplanar with, or in a parallel plane to the side faces, and possibly to the flaps.

Possibly, the bag includes stiffening, which stiffens the bag in the expanded condition.

Possibly, the bag comprises two sheet-like members, which in a disassembled condition may be square or rectangular in shape. Possibly, the two sheet-like members are joined together to form joints along three edges, possibly by welding and/or folding.

Possibly, the joints are continuous along the three edges. Possibly, the joints include side edge joints and an end edge joint. Possibly, the side edge joints extend at or towards a midpoint of the end edge joint.

Possibly, the two sheet-like members comprise part of a single large sheet-like member and may be formed by folding.

Possibly, the joints comprise the stiffening. Possibly, the stiffening biases the bag to the expanded condition.

Possibly, to form the flaps, the bag is located over a former, which may have a substantially square or rectangular cross section, which may correspond in size and shape to the end face.

Possibly, the flaps are substantially triangular in shape.

Possibly, one or more of the joints extend across the end face and the flaps.

Possibly, the sheet-like members are formed of a flexible material, which may comprise fibres, which may be bonded together. Possibly, the sheet-like material comprises paper or a plastics material and may comprise polyester, polypropylene, viscose or similar.

According to a second aspect of the present invention, there is provided a method of controlling pollination of a plant, the method including providing a plant pollination control bag, wherein, in an expanded condition, the bag defines a space in which a plant part is receivable, the bag defining an opening through which the plant part passes into the space, wherein, in the expanded condition, the bag has an end face which is substantially square or rectangular in shape.

According to a third aspect of the present invention, there is provided a method of forming a plant pollination control bag, wherein, in an expanded condition, the bag defines a space in which a plant part is receivable, the bag defining an opening through which the plant part passes into the space, wherein, in the expanded condition, the bag has an end face which is substantially square or rectangular in shape.

Possibly, the bag includes any of the features described in any of the preceding statements or following description. Possibly, the method includes any of the steps described in any of the preceding statements or following description.

### BRIEF DESCRIPTION OF THE DRAWINGS

Embodiments of the present invention will now be described, by way of example only, and with reference to the accompanying drawings, in which:—

FIG. 1 is a perspective view of a plant pollination control bag in an expanded condition;

FIG. 2 is another perspective view of the plant pollination control bag in the expanded condition;

FIG. 3 is yet another perspective view of the plant pollination control bag in the expanded condition;

FIG. 4 is a perspective view of the plant pollination control bag in a flat condition;

FIG. 5 is a perspective view of the plant pollination control bag in the expanded condition, similar to the view of FIG. 2, with opening edges drawn together to restrict an opening;

FIG. 6 is a perspective view of the plant pollination control bag in the expanded condition, similar to the view of FIG. 1, with the opening edges drawn together to restrict an opening;

FIGS. 7 to 11 are schematic views of stages in the formation of the plant pollination control bag.

FIGS. 12 to 16 are schematic views of stages in the formation of a second plant pollination control bag; and

FIG. 17 is a perspective view of the second plant pollination control bag in an expanded condition.

### DETAILED DESCRIPTION OF THE PREFERRED EMBODIMENTS

In the figures, fold lines are shown as dotted lines.

FIGS. 1 to 11 show a plant pollination control bag 10. In an expanded condition, the bag 10 defines a space 12 in which a plant part is receivable. The bag 10 defines an opening 14 through which the plant part passes into the space 12. In the expanded condition, the bag 10 has an end face 16 which is substantially square or rectangular in shape.

The end face 14 is opposite the opening 14, and, in the expanded condition, lies in a plane substantially parallel to the opening 14.

The opening 14 is defined by opening edges 68.

The bag 10 is movable between the expanded condition and a flat condition, in which the bag 10 is substantially flat.

The bag 10 includes reinforcement arrangements 18, which hold the bag 10 in the expanded condition. The bag 10 includes two reinforcement arrangements 18, which are located on opposite sides of the bag 10. Each reinforcement arrangement 18 reinforces an opposite reinforcement edge 20 of the end face 16.

Each reinforcement arrangement 18 includes a substantially triangular flap 22, which in the expanded condition is folded outwardly from the respective reinforcement edge 20 of the end face 16, away from the end face 16 towards a side face 50 of the bag 10.

Each reinforcement arrangement 18 includes a fastening 24, which fastens the flap 22 to the respective side face 50 of the bag 10. The fastening 24 could comprise, for example, a weld, adhesive, a rivet, a staple or a stud, and could be formed by welding, gluing, riveting, stapling or studding.

In the expanded condition, the bag 10 comprises four side faces 50. In the flat condition, the bag 10 comprises two side faces 40. The flat condition side faces 40 each comprise one of the expanded condition side faces 50 between two halves of opposite ones of the expanded condition side faces 50.

In the flat condition, the end face 16 is coplanar with, or in a parallel plane to the side faces 40 and the flaps 22.

With reference to FIGS. 7 to 11, the bag 10 is formed as follows. The bag 10 comprises two sheet-like members 28. The sheet-like members 28 are formed of a flexible material which comprises fibres, which are bonded together. The material comprises a plastics material and could comprise polyester, polypropylene, viscose or similar.

In a disassembled condition, the sheet-like members 28 are square or rectangular in shape. The sheet-like members 28 are joined together to form continuous joints 34 along three edges 38, for example by welding and/or folding.

In one example, as shown in FIG. 7, the two sheet-like members 28 are initially separate and three pairs of edges 38 are welded together by forming edge welds 32.

In another example, as shown in FIG. 8, the two sheet-like members 28 are formed by folding a single larger sheet-like member 44 along a bisecting fold 62, forming an edge fold 30. The other two pairs of edges 38 are then welded to form edge welds 32.

The edge welds 32 form relatively stiff strip-like weld area portions 48.

To form the flaps 22, the bag 10 is opened to the expanded condition and located over a former 36, which passes through the opening 14 into the space 12. The former 36 has a substantially square or rectangular cross section, which corresponds closely in size and shape to the end face 16.

With the former 36 inside the bag 10, pressure is applied to the bag 10 against the former 36 to form folds including longitudinal folds 66 and folds which form end face edges 46 and the flaps 22.

In practice, the longitudinal folds 66 are well defined towards the end face 16, but can be less well defined towards the opening 14.

The flaps 22 are then folded down onto the respective side faces 50 and fastened to the side faces 50, for example, by welding. FIG. 11 shows a welder 64 welding one of the flaps 22 to the respective side face 50 with the bag 10 on the former 36.

The joints 34 extend across the middle of the end face 16, the flaps 22 and the side faces 50 below the reinforcement edges 20.

In the example shown, the end face 16 is rectangular and the shorter edges of the rectangle comprise the reinforcement edges 20.

The bag 10 is moved to the flat condition by forming folds including lateral folds 60 which permit the joints 34 on the side faces 50 to move outwardly and the end face 16 to fold down against the side faces 40 in the flat condition.

In use, the bag 10 is stored and transported in the flat condition. At the point of use, the bag 10 is moved to the expanded condition and then located over the plant part so that the plant part passes through the opening 14 and into the space 12.

The joints 34, comprising the edge fold 30 and the relatively stiff weld area portions 48, comprise stiffening 58. The stiffening 58 helps to keep the bag 10 in the expanded condition, and in fact biases the bag 10 to the expanded condition.

In use, the opening edges 68 can be drawn together to restrict the opening 14, as shown in FIGS. 5 and 6. The applicant has found that even with the opening 14 thus restricted, the square or rectangular end face 16, the reinforcement arrangements 18 and the stiffening 58 help keep the bag in the expanded condition.

Advantageously, the square or rectangular end face 16 provides the space 12 with a relatively large cross section area.

A conventional pollination protection bag comprises inwardly folding side gussets, and has a single end joint rather than an end face opposite to the opening. In comparison with the conventional arrangement, the provision of the end face 16 means that it is easier and quicker to move the bag 10 to the expanded condition.

Unlike a conventional bag, in the bag 10 of the invention, there are no inward folds which can protrude into the space 12 and cause damage to the plant.

Unlike a conventional bag, in the bag 10 of the invention, the fastenings 24 ensure that there are no external pockets which can catch the wind and cause damage to the plant and/or displacement of the bag.

In comparison with a conventional pollination protection bag, the bag 10 of the invention has reduced weight. In one example, the applicant found that in a comparison of similar size bags, the closed end of the bag 10 of the invention had a weight reduced by 30% in comparison with the closed end of the conventional bag.

FIGS. 12 to 17 show a second plant pollination control bag 100, which is a second embodiment of the invention, many features of which are similar to those already described in relation to the embodiment of FIGS. 1 to 11. Therefore, for the sake of brevity, the following embodiment will only be described in so far as it differs from the embodiment already described. Where features are the same or similar, the same reference numerals have been used and the features will not be described again.

With reference to FIGS. 12 to 17, the bag 100 is formed as follows.

In one example, as shown in FIG. 12, the two sheet-like members 28 are initially separate. In a first step, two pairs of side edges 38A are welded together by forming side edge welds 32A to form a tube with open ends. The side edge welds 32A are moved together and longitudinal mid sheet folds 70 are then formed along the middle of the two sheet-like members 28 and the tube then flattened to permit an end edge weld 32B to be formed joining the end side edges 38B. In this example, one side edge weld 32A is located along the middle of each of the flat condition side faces 40 and the longitudinal mid sheet folds 70 are now located at each side edge of the flat condition side faces 40.

In another example, as shown in FIG. 13, the two sheet-like members 28 are formed by folding a single larger sheet-like member 44 along a bisecting fold 62, forming an edge fold 30. Two side edges 38A are welded together by forming a side edge weld 32A to form a tube with open ends. The edge fold 30 and the side edge weld 32A are moved together and longitudinal mid sheet folds 70 are then formed along the middle of the two sheet-like members 28 to permit an end edge weld 32B to be formed joining the end side edges 38B. In this example, the side edge weld 32A is located along the middle of one of the flat condition side faces 40, the bisecting fold 62/end fold 30 is located along the middle of the other of the flat condition side faces 40 and the longitudinal mid sheet folds 70 are now located at each side edge of the flat condition side faces 40.

FIGS. 14 to 17 show the further development of the example shown in FIG. 13.

As previously, to form the flaps 22, the bag 100 is opened to the expanded condition and located over a former 36 and pressure is applied to the bag 100 against the former 36 to form folds including longitudinal folds 66 and folds which form end face edges 46 and the flaps 22.

The flaps 22 are then folded down onto the respective side faces 50 and fastened to the side faces 50, for example, by welding.

The joints 34 extend across the middle of the end face 16 and the flaps 22 and one or both of the side faces 50 not below the reinforcement edges 20. The joint formed by the side edge welds 32A extends to or towards a midpoint of the joint formed by the end edge weld 32B. The joint formed by the end fold 30 also extends to or towards the midpoint of the joint formed by the end edge weld 32B. In the bag produced by development of the example shown in FIG. 12 the end fold 30 is replaced by the other end edge weld 32B.

Advantageously, this embodiment provides an additional longitudinal line of stiffening 58 relative to the previous embodiment, which aids movement of the bag 100 to, and retention of the bag 100 in, the expanded condition.

Various other modifications could be made without departing from the scope of the invention. The bag could be of any suitable size and shape, and could be formed of any suitable material, within the scope of the specific definitions herein.

The bag 10 could be formed of paper. The fastenings could be of any suitable type, formed in any suitable way.

The end face could be square or rectangular in shape. The reinforced edges could be the longer edges or the shorter edges of the rectangular shape. The process of forming could be different to that described. For example, material could be taken from a roll rather than pre-cut.

There is thus provided a plant pollination control bag with a number of advantages over conventional arrangements. The bag can be simply formed. The bag can stored and transported in a flat condition. The bag can be easily moved to the expanded condition, is lighter, and is stable in the expanded condition, reducing damage to plants during placement and removal.

What is claimed is:

1. A plant pollination control bag, wherein, in an expanded condition, the bag defines a space in which a plant part is receivable, the bag defining an opening through which the plant part passes into the space, wherein, in the expanded condition, the bag has an end face which is substantially square or rectangular in shape.

2. The bag according to claim 1, in which the end face is opposite the opening, and wherein in the expanded condition, the end face lies in a plane parallel to the opening.

3. (canceled)

4. The bag according to claim 1, in which the bag is movable between the expanded condition and a flat condition, in which the bag is substantially flat.

5. The bag according to claim 1, in which the bag includes reinforcement arrangements, which hold the bag in the expanded condition.

6. The bag according to claim 5, in which the bag includes two reinforcement arrangements, which are located on opposite sides of the bag, and wherein each reinforcement arrangement reinforces an opposite reinforcement edge of the end face.

7. (canceled)

8. The bag according to claim 6, in which each reinforcement arrangement includes a flap.

9. The bag according to claim 8, in which each flap folds outwardly from the respective reinforced edge of the end face, away from the end face towards a side face of the bag.

10. The bag according to claim 9, in which each reinforcement arrangement includes a fastening, which fastens the flap to the respective side face of the bag.

11. The bag according to claim 10, in which the fastening comprises a weld, adhesive, a rivet, a staple or a stud, and may be formed by welding, gluing, riveting, stapling or studding.

12. The bag according to claim 4, in which, in the expanded condition, the bag comprises four side faces; and in the flat condition, the bag comprises two side faces and the end face is coplanar with, or in a parallel plane to the side faces.

13-14. (canceled)

15. The bag according to claim 8, in which the bag includes stiffening, which stiffens the bag in the expanded condition.

16. The bag according to claim 15, in which the bag comprises two sheet-like members, which in a disassembled condition are square or rectangular in shape and are joined together to form joints along three edges.

17. The bag according to claim 16, in which the joints are formed by welding and/or folding, and wherein the joints are continuous along the three edges.

18. (canceled)

19. The bag according to claim 17, in which the joints include side edge joints and an end edge joint, and wherein the side edge joint extends at or towards a midpoint of the end edge joint.

20. (canceled)

21. The bag according to claim 16, in which the two sheet-like members comprise part of a single large sheet-like member and are formed by folding.

22. The bag according to claim 16, in which the joints comprise the stiffening, and the stiffening biases the bag to the expanded condition.

23. (canceled)

24. The bag according to claim 8, in which, to form the flaps, the bag is located over a former which has a substantially square or rectangular cross section that corresponds in size and shape to the end face.

25-26. (canceled)

27. The bag according to claim 8, in which the flaps are substantially triangular in shape.

28. The bag according to claim 16, in which one or more of the joints extend across the end face and the flaps.

29. The bag according to claim 16, in which the sheet-like members are formed of a flexible material selected from the group consisting of fibers, bonded fibers, paper, plastic, polyester, polypropylene and viscose.

30-34. (canceled)
